(19) United States
(12) Patent Application Publication
(10) Pub. No.: US 2005/0085827 A1
(43) Pub. Date: Apr. 21, 2005

(54) Uterine manipulating device

(76) Inventors: Anselmo Najera G., El Paso, TX (US); Cesar A. Barrientos, CD Juarez (MX)

Correspondence Address: R WAYNE PRITCHARD, 863 VIA ALTA LANE, EL PASO, TX, 79912, US

(21) Appl. No.: 10/686,072

(22) Filed: Oct. 15, 2003

(51) Int. Cl. A61B 17/42

(52) U.S. Cl. 606/119; 128/830

(57) ABSTRACT

An ergonomically designed, low cost uterine manipulator comprising a handle, a shaft in general curvature of a female cervix, a vaginal delineator, and a uterine tip.

### BACKGROUND OF THE INVENTION

1. Field of the Invention

This invention relates to uterine manipulators and more particular to uterine manipulators used in performing laparscopic surgical procedures on the female uterus such as a laparaoscopic hysterectomy, where removal of tissue specimens through a colpotomy incision is recommended.

2. Description of the Prior Art

Changing lifestyles, environment as well as an ever aging female population has increased the medical necessity for female hysterectomies. Conventional hysterectomy surgical procedures generally involved one of the four following approaches: (1) vaginal hysterectomy (“VH”) (removal of the uterus through the vaginal cavity); (2) total abdominal hysterectomy (“TAH”)(removal of the uterus through an abdominal incision); (3); laparscopically assisted vaginal hysterectomy (“LAVH”)(removal of the uterus through the vagina following laparscopic procedure), (4) total laparascopic hysterectomy (“TLH”)(removal of the uterus through small abdominal incisions). The need to reduce the amount of trauma inflicted upon a patient during surgery as well as the time necessary to recover from such surgery has lead to a move away from the most invasive TAH procedure towards the less invasive VH, LAVH and TLH procedures. Unless medical conditions indicate the need for TAH such as when the need to avoid cell spillage through the removal of a tumor exists, the TLH procedure is the most desired because it is the least invasive resulting in less trauma to the patient and consequently shorter hospitalization stays and less recovery time. Notwithstanding the foregoing, the move towards TLH has been somewhat hampered by the complicated and expensive uterine manipulators currently available. A simple, inexpensive, and dependable uterine manipulator, such as that disclosed in the present invention, would no doubt help continue the move towards more TLH procedures. Although the present invention has been designed as an instrument to be used in connection with the TLH procedure, it can also be used with the somewhat more expensive, more invasive LAVH procedure.

In the medical field, it is generally the case that the more complicated surgical procedures as well as the more complicated surgical instruments are more expensive. The more a procedure or an instrument can be simplified, the less it will cost. There is a broad range of surgical procedures which require the manipulation of the uterus. The instruments used in connection with such procedures range from complex to simple and perhaps, more importantly, expensive to affordable. For instance, the simplest manipulator would be one such as that disclosed in U.S. Pat. No. 3,809,091 to Shute, wherein the manipulator is used only to shift the position of the uterus so that surgery can be performed on other parts of the female anatomy. There are no mechanisms in the Shute patent to capture the cervical end of the uterus nor any way to seal the uterus. At the other end of the spectrum, various complex manipulators have been designed which will allow the uterus to be sealed, which enables dye to be retained inside the uterus. U.S. Pat. No. 5,549,563 to Kronner; U.S. Pat. No. 5,464,409 to Mohajer; and U.S. Pat. No. 4,775,362 again to Kronner, as well as the UMI uterine manipulator disclosed on the web page www.surgimed.com/umi.htm, all disclose manipulators which contains a balloon like structure on the tip to be inserted into the uterus. The balloon like structure seals the cervical entrance of the uterus. and does not allow for fluids to leave.

Uterine manipulators used in connection with hysterectomies, unlike those disclosed above, must contain some form of vaginal delineator. The vaginal delineator captures the cervical end of the uterus and allows the surgeon to perform the required colpotomy. For example, U.S. Pat. No. 5,840,077 to Rowden, discloses a “L” shaped complex uterine manipulator with the vaginal delineator identified as item number 96. Unlike the present invention however, the Rowden manipulator contains several balloon like structures designed to seal the uterus (identified as items number 70 and 43) along with a hinged mechanism to rotate the tip of the manipulator. The vaginal delineator contains a mechanism allowing it to lock onto the hinged structure of the manipulator shaft. Further, the Rowden manipulator is not constructed in an ergonomic banana shape but rather is fashioned in the shape of an “L” which necessitates the inclusion of the complicated hinged mechanism referred to above. As can be appreciated by those in the art, a straight uterine manipulator does a very poor job of allowing the surgeon to manipulate the uterus. A uterine manipulator in a shape which mimics the contours of the female vaginal cavity, i.e. a banana shape, does a much better job of allowing the surgeon to easily shift the uterus in any direction desired.

Another uterine manipulator which contains a vaginal delineator is disclosed in U.S. Pat. No. 5,209,754 to Ahluwalia. The vaginal delineator disclosed in the Ahluwalia patent however, is far more complicated the vaginal delineator of the present invention disclosing what can be described as a bowl in a bowl. The cervical end of the uterus is designed to fit into the gap existing as a result of the bowl in bowl structure, apparently, to facilitate improved surgical precision. The HOHL uterine manipulator disclosed at www.karlstorz.com/hm/2 products/2 13/2 13 2 3.htm. also contains a vaginal delneator. The HOHL manipulator, however, does not utilize the ergonomic design of the present invention which does not allow for complete capture of the cervical end of the uterus.

### SUMMARY OF THE INVENTION

It is, therefore, an object of the present invention to provide an ergonomically designed, low cost uterine manipulator. Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of the intra-uterine manipulator fully assembled and ready for surgical intervention.

FIG. 2 is a side view of the shaft.

FIG. 3 is a plan and side view of the handle.

FIG. 4 is a side and plan view of the vaginal delineator.

FIG. 5 is a perspective view of the uterine tip.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

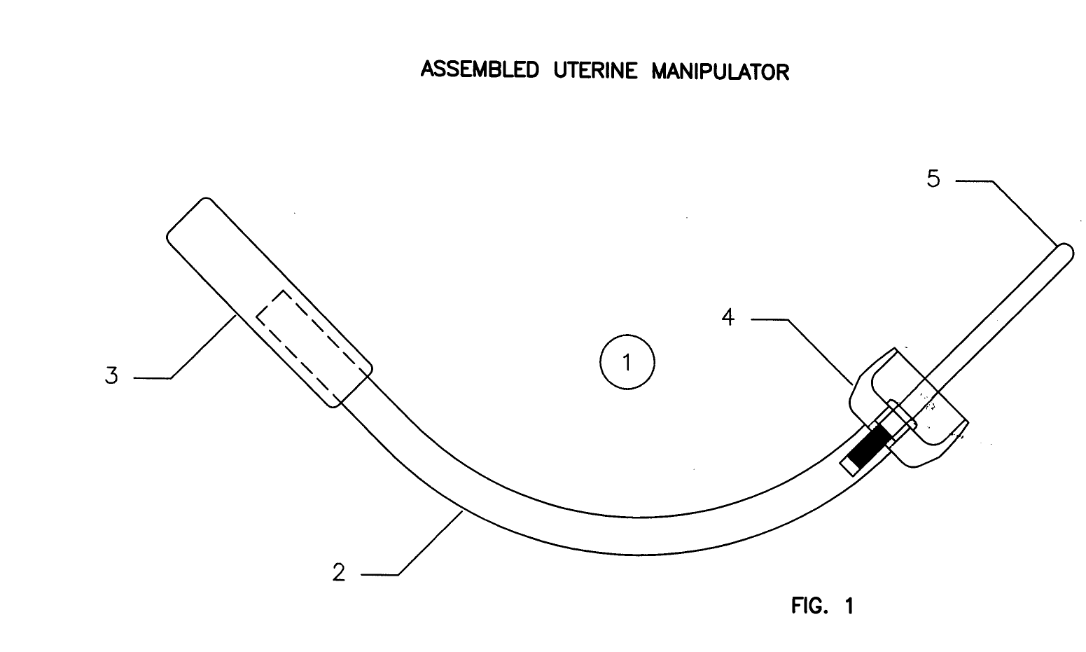

With reference to FIG. 1, a fully assembled uterine manipulating device [1] is disclosed which comprises a handle [3], a shaft in the general curved shape of a female cervix [2], a vaginal delineator [4], and a uterine tip [5]. It has been found that a shaft [2] diameter of between 1.27 and 1.78 cm (0.50 and 0.70 inches) is desired although the most preferred shaft [2] diameter is 1.59 cm (0.625 inches).

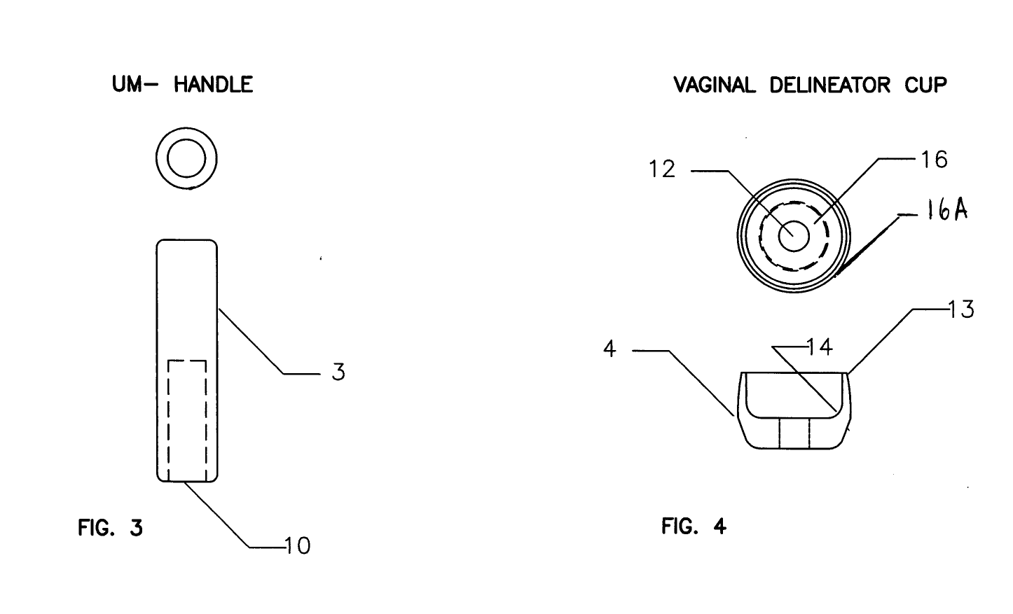

To achieve a more ergonomic design, the diameter of the handle [3] has been increased to a distance slightly greater than the diameter of the shaft [2]. Although any handle diameter of between 2.03 and 3.8 cm (0.80 to 1.50 inches) will achieve the desired ergonomic design, a handle [3] diameter of 2.54 cm (1 inch) is optimum. While the shaft [2], handle [3], uterine tip [5] and vaginal delineator [4] could be of one piece construction, as can be seen from FIGS. 1-5, the present invention discloses each of the foregoing to be separate and distinct parts. Although any conventional means of attachment such as pressure fit, epoxy or glue could be used to attach the handle [3] to the shaft [2], as shown in FIGS. 1 and 3, the handle [3] has been adapted [10] to allow for the insertion of one end of the shaft [2]. In the preferred embodiment of the present invention, the handle [3] and shaft [2] are threaded to allow for removable fixation of the handle [3] to the shaft [2].

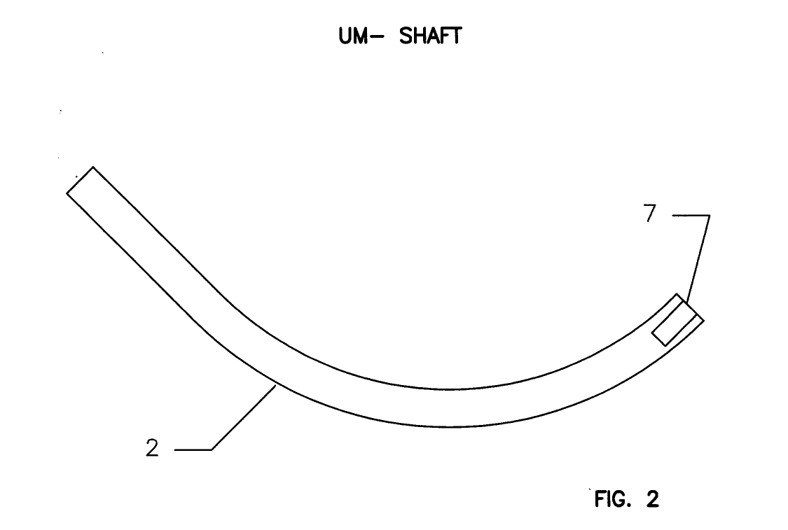

FIG. 2 details that the shaft [2] is constructed in a curved shape. The curved shape resembles the natural shape of the female vaginal cavity and enables the operator to grab the assembled uterine manipulator [1] in a position ergonomically efficient, thus reducing fatigue for the surgeon and/or extending the amount of time a surgeon can use the manipulator before becoming fatigued. The term “ergonomically efficient” refers to the fact that the shaft [2] curvature allows the manipulator to move the uterus in all directions with ease. The radius of curvature for the shaft [2] can be any distance between 12.70 and 20.32 cm (5 and 8 inches), preferably a constant 15.24 cm (6 inches).

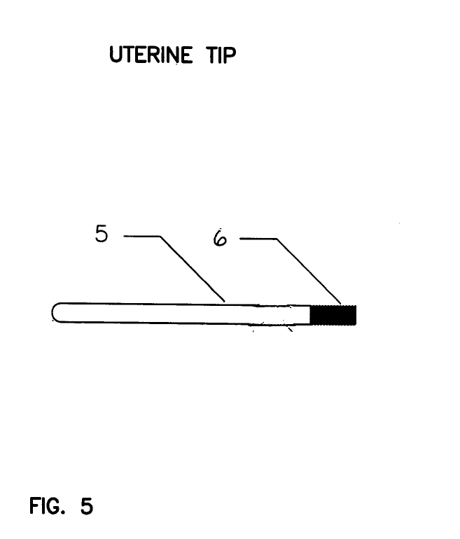

Although the vaginal delineator [4] shown in FIG. 4, can be made from any material that is non-conductive in order to allow for colpotomy using bipolar cautery, the vaginal delineator [4] of the present invention is made of low porosity, high temperature resistant acrylic. As shown in FIG. 4, the vaginal delineator [4] is made in the general shape of a cup is given a rounded [15] shape with radiused [13, 14] corners to reduce or eliminate the risk of damage to the inside of the vagina when the uterine manipulator [1] is inserted. The Vaginal delineator [4] of the present invention can be made of various diameters to accommodate varying sizes of the female uterus such any diameter between 3.81 and 6.35 cm (1.50 and 2.5 inches), preferably in standard diameters of 4.06, 4.57 and 5.08 cm (1.60, 1.80 and 2 inches). The inside [16] and outside [16A] surfaces of the vaginal delineator [4] are polished to reduce the coefficient of friction; enabling an easier vaginal penetration. Attachment of the vaginal delineator [4] to the shaft [2] can be achieved using any conventional means such as pressure fit, epoxy or glue, however, in the present invention, as can be seen in FIGS. 2 and 4, an inside portion [12] of the vaginal delineator [4] of diameter sufficient to allow centered insertion of the shaft [2] has been evacuated.

The uterus varies in size depending upon the anatomy of the patient. In order to accommodate different sized uteruses, the uterine tip [5], shown best in FIG. 5, can be made in varying lengths between 2.54 and 12.70 cm (1 and 5 inches), preferably in standardized lengths of 5.08, 7.62 and 10.16 cm (2, 3, and 4 inches). The uterine tip [5] should be of a diameter slightly smaller than the shaft [2] in a range of between 0.64 and 1.14 cm (0.25 and 0.45 inches). It has been found that a uterine tip [5] diameter of 0.95 cm (0.375 inches) is the most optimum. The uterine tip [5] is then attached to the shaft [2] using any conventional means such as pressure fit, epoxy or glue. As shown in FIG. 5, in the preferred embodiment of the present invention, the uterine tip [5] is threaded so as to be inserted in and through the evacuated portion [12] of the vaginal delineator [4]. As shown in FIG. 2, the uterine tip [5] end of the shaft [2] has been adapted [7] to allow for insertion of the uterine tip [5], which once inserted will allow for affixation of the uterine tip [5] to the shaft [2]. Once the uterine tip [5] is affixed to the shaft [2], the vaginal delineator is positionally secured.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. A uterine manipulating device comprising a shaft in the general curved shape of a female cervix having a handle end of slightly larger diameter than the shaft; a uterine tip end of slightly smaller diameter than the shaft; a vaginal delineator; and a means for attaching the vaginal delineator to the shaft.

2. A uterine manipulating device comprising a shaft of diameter D1, generally curved in the shape of a female cervix wherein the tip of one end of said shaft has a reduced diameter D2 and onto which a vertical delineator of diameter D3 is affixed at a distance of L1 from said end.

3. The uterine manipulating device as in claim 2 wherein the radius of curvature of the shaft is between 12.70 and 20.32 cm (5 and 8 inches).

4. The uterine manipulating device as in claim 3 wherein the radius of curvature of the shaft is a constant 15.24 cm (6 inches).

5. The uterine manipulating device of claim 2 wherein D1 is a diameter of between 1.27 and 1.78 cm (0.50 and 0.70 inches); D2=D−0.64 cm (0.25 inches) and L1 is a distance of between 2.54 and 12.70 cm (1 and 5 inches).

6. The uterine manipulating device of claim 5 wherein D1 is 1.59 cm (0.625 inches); D2=0.95 cm (0.375 inches) and L1 is a distance between 5.08 and 10.16 cm (2 and 4 inches).

7. The uterine manipulating device as in claim 2 wherein the diameter of the vertical delineator D3 is a distance of between 3.81 and 6.35 cm (1.5 and 2.5 inches).

8. The uterine manipulating device as in claim 2 wherein the diameter of the vertical delineator D3 is a distance of between 4.06 and 5.08 cm (1.6 and 2.0 inches).

9. A uterine manipulating device comprising a shaft of diameter D1, generally curved in the shape of a female cervix; a handle of diameter D2; a uterine tip of diameter D3; and onto which a vertical delineator of diameter D4 is affixed at a distance of L1 from the end of the uterine tip.

10. The uterine manipulating device as in claim 9 wherein D2 is a distance of between 2.03 and 3.8 cm (0.80 and 1.50 inches).

11. The uterine manipulating device as in claim 9 wherein D2 is a distance of 2.54 cm (1 inch).

12. The uterine manipulating device as in claim 9 wherein the shaft wherein the radius of curvature of the shaft is between 12.70 and 20.32 cm (5 and 8 inches).

13. The uterine manipulating device as in claim 12 wherein the radius of curvature of the shaft is a constant 15.24 cm (6 inches).

14. The uterine manipulating device of claim 9 wherein D1 is a diameter of between 1.27 and 1.78 cm (0.50 and 0.70 inches); D3=D1−0.64 cm (0.25 inches) and L1 is a distance of between 2.54 and 12.70 cm (1 and 5 inches).

15. The uterine manipulating device of claim 9 wherein D1 is 1.59 cm (0.625) inches; D3=0.95 cm (0.375 inches) and L1 is a distance between 5.08 and 10.16 cm (2 and 4 inches).

16. The uterine manipulating device as in claim 9 wherein the diameter of the vertical delineator D4 is a distance of between 3.81 and 6.35 cm (1.5 and 2.5 inches).

17. The uterine manipulating device as in claim 9 wherein the diameter of the vertical delineator D4 is a distance of between 4.06 and 5.08 cm (1.6 and 2.0 inches).

18. The uterine device as in claim 9 wherein one end of the handle and one end of the shaft are threaded so as to be removeably attached to each other.

19. The uterine device as in claim 9 wherein one end of the uterine tip and the end of the shaft opposite to the handle are threaded so as to be removeably attached to each other.
